(19) United States
(12) Patent Application Publication
(10) Pub. No.: US 2002/0127233 A1
(43) Pub. Date: Sep. 12, 2002

(54) Method for inhibiting inflammation in immune privileged sites using Fas ligand fragments

(76) Inventors: Bing Zhu, Vancouver (CA); Max S. Cynader, West Vancouver (CA); Donald W. Paty, Vancouver (CA); Liqing Luo, Vancouver (CA)

Correspondence Address: GARY CARY WARE & FRIENDENRICH LLP, 4365 EXECUTIVE DRIVE, SUITE 1600, SAN DIEGO, CA, 92121-2189, US

(21) Appl. No.: 09/927,110

(22) Filed: Aug. 10, 2001

Related U.S. Application Data
(60) Provisional application No. 60/224,016, filed on Aug. 10, 2000.

(51) Int. Cl. A61K 39/00

(52) U.S. Cl. 424/184.1; 424/185.1

(57) ABSTRACT

The present invention is directed to a method of modulating inflammation within an immune privileged site in an animal by introducing an effective amount of a Fas ligand fragment comprising the extracellular domain of a full length Fas ligand, a derivative thereof, or a nucleic acid encoding the Fas ligand fragment, behind the blood-tissue barrier of the immune privileged site. In one embodiment the invention pertains to methods of modulating inflammation in the central nervous system generally, at specific lesions in the central nervous system, anterior chamber of the eye, testis, placenta and other immune privileged sites in a mammal. The FasL fragments used in the method of the present invention contain the extracellular domain of FasL and are soluble. The method of the present invention comprises the step of directly administering the FasL fragment, or derivative thereof, or a composition comprising the FasL fragment, or derivative thereof, behind the blood-tissue barrier of the immune privileged site.

### RELATED APPLICATIONS

This application claims priority to U.S. Provisional Application No. 60/224,016 filed Aug. 10, 2000, which is herein incorporated by reference in its entirety.

### FIELD OF THE INVENTION

This invention relates generally to the modulation of inflammation in immune-privileged sites in mammals by directly introducing rFasL ligand behind the immune barrier.

### BACKGROUND

FasL has been shown to be important in maintaining immune privilege in the anterior chamber of the eye (Griffith T. S. et al. (1995) Science. 270:1189-1192), testis (Bellgrau D, et al. (1995) Nature 377:630-632; Takeda Y., et al. (1998) Diabetologia. 41:315-321) and placenta (Hunt J. S., et al. (1997) J. Immunol. 158: 4122-4128; Uckan D. et al. (1997) Mol Hum Reprod. 3:655-662). Two types of mutant mice, lpr (no Fas receptor expression) and gld (no functioning FasL expression), show the breakdown of immune privilege in these sites. In relation to this, FasL is essential in activation-induced cell death (AICD) (Dhein J. et al. (1995) Nature 373:438-441; Brunner T. et al. (1995) Nature 373: 441-444), in which the activation of T cells sensitizes them to apoptosis. AICD may be important in limiting the intensity of an inflammatory response and in removing the inflammation after the immune function has been fulfilled (Depraetere V. et al. (1997) Semin Immunol. 9:93 -107). In some experimental conditions, the activated B cells, macrophages, and even the “bystander” monocytes and neutrophils have also been found vulnerable to FasL-induced apoptosis (Ashany D. et al. (1995) Proc Natl Acad Sci USA. 92:11225-11229; Kiener P. A. et al. (1997) J Immunol 159:1594-1598; Watanabe D. et al (1995) Int Immunol. 7:1949-1956; Brown S. B. et al. (1999) J Immunol 162:480-485). Furthermore, overexpression of FasL in tissues targeted by autoimmunity has been shown to reduce the disease severity in autoimmune arthritis (Zhang H. et al. (1997) J Clin Invest. 100:1951-1957; Okamoto K. et al. (1998) Gene Ther. 5:331-338) and thyroiditis (Batteux F. et al. (1999) J Immunol. 162:603-608; Batteux F. et al. (2000) J Immunol. 164:1681-1688) models.

In the recovery phase of EAE, the infiltrated Vβ8.2+ T cells, B cells and macrophages have high frequencies of Fas and FasL expression, and are highly vulnerable to apoptosis (McCombe P. A. et al. (1996) J Neurol Sci. 139:1-6; White C. A. et al. (2000) J Autoimmun. 14:195-204; Kohji T. et al. (2000) J Neuroimmunol. 106:165-171). This suggests that the upregulation of the Fas system in the CNS is an endogenous mechanism to resolve the CNS autoimmune inflammation. However, this upregulation occurs during the EAE course, and has no effect in inhibition of the development of EAE. In contrast to other immune privileged sites, constitutive FasL expression is low in the CNS (French L. E. et al. (1996) J Cell Biol. 133:335-343; Xerri L. et al. (1997) Mol Pathol. 50:87-91).

The ability of FasL to destroy activated T cells suggests that it has potential as an immunosuppressive drug. However, FasL is likely to be highly toxic when injected into animals and humans, because it will induce apoptosis of other cells expressing Fas in addition to T cells, for example liver cells. Indeed, an agonistic antibody to murine Fas rapidly kills mice after intraperitoneal administration by causing massive necrosis of the liver, presumably mediated through apoptosis of hepatocytes via Fas (J. Ogasawara, Nature 364:806, 1993).

One attempt to overcome this difficulty involved the use of FasL fusion proteins that have specific cytotoxicity to autoimmune T cells because they comprise a polypeptide capable of specifically binding an antigen or cell surface marker (U.S. Pat. No. 6,046,310).

This background information is provided for the purpose of making known information believed by the applicant to be of possible relevance to the present invention. No admission is necessarily intended, nor should be construed, that any of the preceding information constitutes prior art against the present invention. Publications referred to throughout the specification are hereby incorporated by reference in their entireties in this application.

### SUMMARY OF THE INVENTION

The present invention pertains to a method for inhibiting inflammation in immune privileged sites using Fas lignd fragments.

One aspect of the present invention provides a method of inhibiting inflammation within an immune privileged site in an animal by introducing an effective amount of a Fas ligand (FasL) fragment comprising the extracellular domain of a full length Fas ligand, or a derivative thereof, behind the blood-tissue barrier of the immune privileged site.

In one aspect the present invention pertains to the use of FasL fragment to potentiate the immune privilege of CNS, and prevent the development of acute EAE by eliminating activated autoreactive T cells and/or macrophages during their infiltration into the CNS.

Another aspect of the present invention pertains to the use of FasL fragment to create an immune privileged site in an animal in need of such therapy.

### BRIEF DESCRIPTION OF THE FIGURES

FIG. 1 demonstrates intrathecal rFasL infusion reduced the degree of inflammation, the numbers of ED1+ cells and OX19+ cells in both the meningeal, perivascular areas and the spinal cord parenchyma. Panels A, C, and E are micrographs of HE staining, ED1, and OX19 immunostaining of control-infused EAE rat LSSC sections. Panels B, D, and F are those of corresponding staining of rFasL-infused EAE-prevented rat LSSC sections. Scale bar=35 μm.

FIG. 2 depicts the quantitation of inflammation and immunostaining positive cells. Panels A and B show the counts of inflammatory foci and the scores of inflammation that were quantitated from immunized-only, control-infused and rFasL-infused rat LSSC sections. Panel C shows the quantitation of ED1, OX19, OX42, W3/13, W3/25 immunostaining positive cells in control-infused and rFasL-infused rat LSSC sections. (*p<0.05)

FIG. 3 demonstrates Fas receptors are highly expressed in ED1+ and OX19+ cells in EAE rat LSSC, and are constitutively expressed on astrocytes, neurons and oligodendrocytes in normal rat LSSC. RFasL infusion greatly reduced Fas+ cells, and the remaining Fas+ cells showed same pattern as in normal rat LSSC. Panels A, B, C, D, and E show double immunostaining with Fas antibody (brown) and OX19, ED1, Rip, GFAP, and SMI-32 antibodies (grey) respectively. Panels F and G compare the anti-Fas immunostaining in a control-infused EAE rat LSSC section and a rFasL-infused EAE-prevented rat LSSC section. Scale bar for panels A-E=5 μm, and scale bars for panels F & G=23 μm.

FIG. 4 demonstrates that in vitro rFasL treatment induced morphologically apoptotic death in MBP-specific T line cells. T line cells were in the second round of MBP stimulation with gamma-irradiated thymocytes as APCs. After 24 hours of MBP stimulation, control cells were not treated, and the duplicate culture was treated with 200 ng/ml rFasL. Sixteen hours later, non-treated T blasts (panel A) were almost confluent and were much bigger in sizes compared with co-cultured gamma-irradiated thymocytes. In the dish that was treated with rFasL, (panel B) most T blasts were dead or dying, and very few remained normal morphology. Many dying T blasts show typical morphologic apoptotic changes.

FIG. 5 depicts Annexin V-FITC/PI staining and flow cytometry and shows that rFasL dose-dependently induced apoptosis in MBP-activated T blasts. Because virtually all gamma-irradiated thymocytes were positive in PI staining after two days in culture (panel A), most PI-negative cells in co-culture were the bigger T blasts (panels B & C). With the doses of rFasL from 0 to 25 ng/ml, 50 ng/ml, 100 ng/ml and 200 ng/ml, T blasts double negative for Annexin V-FITC and PI staining among total cells decreased significantly from 42.9% to 20.5%, 10.5%, 6.1% and 3.7% respectively. (Panels D to H) The results were typical for three separate experiments.

FIG. 6 demonstrates that in vitro treatment of MBP-specific T line cells with 200 ng/ml rFasL for 16 hours abrogated the encephalitogenicity in T line cells. 2×10⁶ non-treated T blasts were intravenously transferred into each of four naive Lewis rats, which invariably developed 3⁰ EAE, (panel A) and reduced over 30 grams in body weight during EAE. (Panel B) In contrast, cells from duplicate culture but treated with rFasL were collected and transferred in the same way as non-treated cells but could not transfer EAE at all.

FIG. 7 demonstrates that RFasL treatment dose-dependently induced cell death in activated macrophages. Peritoneal inflammatory macrophages were activated in vitro with 100 U/ml IFN-gamma for 24 hours and then triggered with 200 ng/ml of LPS. RFasL, either alone or with the anti-FLAG antibody that cross-links rFasL, was added at the same time as LPS. MTT assay was performed 16 hours later. The results were typical for three separate experiments.

FIG. 8 demonstrates rFasL dose-dependently potentiated the inhibition of T cell proliferation by rat CSF. Zero to 50% (V/V) rat CSF was included from the start of T cell proliferation experiment. RFasL was added 24 hrs later. [³H]-thymidine was added another 24 hours later, and cells were harvested further 16 hours later. The results were representative for three separate experiments.

FIG. 9 shows TUNEL staining in LSSC sections from control-infused (panels A, C) and rFasL-infused (panels B, D) rats on day 12 dpi. TUNEL+ cells are in brown and the cell nuclei are counterstained in green with methyl green. In a LSSC section from a control-infused, 3⁰ EAE rat, TUNEL+ cells appear mostly in the parenchyma of LSSC (panel A), but are rare in the inflammatory meningeal and perivascular areas (panels A & C). In a LSSC section from a rFasL-infused, EAE-free rat, the TUNEL+ cells are less common in both the parenchyma and meningeal/perivascular areas, where inflammation is minimal (panel B). However, in another section from a rFasL-infused, 1⁰ EAE (complete tail paralyzed) rat, the TUNEL+ cells are greatly increased in the meningeal and perivascular areas of LSSC, where mild inflammation is present (panel D). Scale bar=30 μm.

FIG. 10 demonstrates the prevention of EAE by rFasL infusion was not due to the suppression of systemic immune response to MBP. Panel A compares the MBP-induced DTH responses in immunized only, control-infused, and rFasL-infused rats on 12 dpi, which do not show any significant differences among these three groups of animals. Panel B compares the MBP-induced T cell proliferation between the control infused, and the rFasL infused rats on both 10 dpi and 12 dpi, which again does not show any significant differences. (p>0.05)

FIG. 11 demonstrates that RFasL infusion did not damage astrocytes, oligodendrocytes or the myelin structure in rat LSSC. Panels A and C are micrographs showing anti-GFAP and Rip staining in normal non-immunized rat LSSC sections. Panels B and D are corresponding staining in rFasL-infused rat LSSC sections, which show that astrocytes and oligodendrocytes are present in same densities and staining patterns as in panel A and C. Luxol fast blue staining of rFasL-infused LSSC sections (panel E) does not show any area with myelin loss. Toluidine blue staining on semi-thin LSSC sections (panel F) shows intact myelin structure and normal myelin thickness. Scale bar for panels A-E=23 μm, and the scale bar for panel F=12 μm.

FIG. 12 depicts the amino acid sequence of a full length human Fas ligand (GenBank accession number P48023)

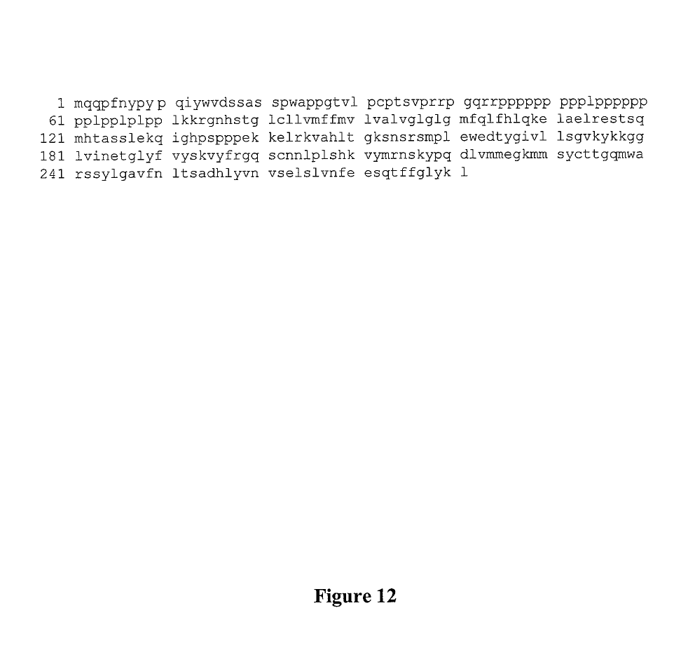

### DETAILED DESCRIPTION OF THE INVENTION

The present invention is directed to a method of modulating inflammation within an immune privileged site in an animal by introducing an effective amount of a Fas ligand fragment comprising the extracellular domain of a full length Fas ligand, a derivative thereof, or a nucleic acid encoding the Fas ligand fragment, behind the blood-tissue barrier of the immune privileged site. In one embodiment the invention pertains to methods of modulating inflammation in the central nervous system generally, at specific lesions in the central nervous system, anterior chamber of the eye, testis, placenta and other immune privileged sites in a mammal. The FasL fragments used in the method of the present invention contain the extracellular domain of FasL and are soluble. The method of the present invention comprises the step of directly administering the FasL fragment, or derivative thereof, or a composition comprising the FasL fragment, or derivative thereof, behind the blood-tissue barrier of the immune privileged site. The term “behind the blood-tissue barrier,” as used herein, implies administration of the FasL fragment, or derivative thereof, or a composition comprising the FasL fragment, or derivative thereof into the immune privileged site.

It would be readily apparent to a worker skilled in the art that there are a variety of diseases and disorders that can be treated using the method of the present invention. Provided below is a non-limiting list of exemplary diseases that may be treated according to the present invention:

1. In the eye:

1) rejection of corneal transplantation.

2) anterior uveitis caused or secondary to rheumatoid arthritis, herpes simplex virus infection, herpes zoster infection, ankylosing spondylitis, postsurgical anterior uveitis, Reiter's syndrome, trauma, inflammatory bowel disease, or idiopathic.

3) posterior uveitis secondary to systemic lupus erythematous, sympathetic ophthalmia, systemic tuberculosis infection.

4) intermediate uveitis secondary to sarcoidosis, multiple sclerosis, inflammatory bowel disease.

5) optic neuritis: idiopathic or secondary to multiple sclerosis.

6) glaucoma: it is suggested to have inflammation in pathogenesis.

7) ocular sarcoidosis: a panuveitis, occasional involvement of the optic nerve and retinal blood vessels.

8) sympathetic ophthalmia: an inflammatory reaction in the second eye after the other has been damaged by penetrating injury.

2. In the CNS:

1) multiple sclerosis, optic neuritis.

2) acute disseminated encephalomyelitis: an acute disease that may follow a viral exanthem. There is an interval between the viral infection and the CNS disease. It may be postinfectious, e.g. measles, or postvaccinal e.g. after rabies and smallpox vaccine immunization.

3) acute transverse myelitis: may occur after viral infections, tumors, vascular malformations, development abnormalities, Vitamin B12 deficiency, or degeneration.

4) acute inflammatory demyelinating polyneuropathy: that follows acute viral infections or enteric infection with Campylobacter jejuni infection.

5) chronic demyelinating polyneuropathies.

6) paraneoplastic cerebellar degeneration: occurring as an indirect effect of systemic cancer.

7) limbic encephalitis: occurs either as a papaneoplastic syndrome, or without and underlying cause.

8) amyotrophic lateral sclerosis.

9) Alzheimer's disease.

10) Immunological features of stroke.

11) other CNS diseases with autoimmune features: such as Rasmussen's encephalitis and “stiff-man” syndrome.

3. reproductive system:

1) infertility induced by autoimmune diseases of the testis and ovary.

2) recurrent spontaneous abortion.

3) orchitis: granulomatous orchitis, chronic orchitis, and Malakoplakia orchitis.

In an alternative embodiment, the method of the present invention can be used in the treatment of other organ directed inflammatory diseases, wherein it is beneficial to create an immune privilege site. One could administer FasL fragments (or nucleic acids encoding the FasL fragments) and derivatives thereof and compositions containing them to an organ, for example, thereby creating an immune privileged site within the blood-tissue barrier of the organ. The immune privileged site may be permanent or temporary, depending upon the disorder being treated and on the mode of administration; gene therapy may result in long term existence of the immune privileged site. For example, this method can be used in the treatment of autoimmune thyroid disease and arthritis, etc.

In one embodiment of the present invention the FasL fragments and derivatives thereof and compositions containing them are used for the inhibition of autoimmune inflammation. In a related embodiment the autoimmune inflammation is CNS autoimmune inflammation, for example, multiple sclerosis.

Active Fragments of FasL

According to one embodiment of the present invention the active fragments of FasL that are used to inhibit inflammation contain the extracellular domain of FasL, are soluble and are able to potentiate immune privilege when administered to a mammal. In one embodiment of the present invention the FasL fragment comprises amino acids 103-281 of the full length FasL.

A worker skilled in the art would readily appreciate that the FasL fragments, and derivatives thereof, that are used in the method of the present invention may be from any animal. In one embodiment of the present invention the FasL fragment is from a mammalian FasL molecule. The mammalian FasL may be from an animal such as, but not limited to, human, bovine, pig, rat or mouse. Sequences of such FasL molecules are readily available from public databases such as GenBank.

Preparation of FasL Fragments

The fragments of FasL according to the present invention can be prepared using a variety of techniques known to those skilled in the art, including via recombinant DNA technology, synthetic techniques or by enzymatic or chemical cleavage of full length FasL.

Purification of the FasL fragments, or derivatives thereof, is carried out by standard methods including chromatography (e.g., ion exchange, affinity, and sizing column chromatography), centrifugation, differential solubility, hydrophobicity, or by any other standard technique for the purification of proteins.

The FasL fragments of the present invention may be prepared from cell extracts, or through the use of recombinant techniques. In general, FasL fragments according to the invention can be produced by transformation (transfection, transduction, or infection) of a host cell with all or part of a FasL fragment-encoding nucleic acid in a suitable expression vehicle. Suitable expression vehicles include: plasmids, viral particles, and phage. The entire expression vehicle, or a part thereof, can be integrated into the host cell genome. In some circumstances, it is desirable to employ an inducible expression vector, e.g., the LACSWITCH™ Inducible Expression System (Stratagene, LaJolla, Calif.).

Those skilled in the field of molecular biology will understand that any of a wide variety of expression systems can be used to provide the recombinant protein. The precise host cell used is not critical to the invention. The FasL fragments can be produced in a prokaryotic host (e.g., E. coli or B. subtilis) or in a eukaryotic host (e.g., Saccharomyces or Pichia; mammalian cells, e.g., COS, NIH 3T3, CHO, BHK, 293, or HeLa cells; or insect cells).

Proteins and polypeptides can also be produced by plant cells. For plant cells viral expression vectors (e.g., cauliflower mosaic virus and tobacco mosaic virus) and plasmid expression vectors (e.g., Ti plasmid) are suitable. Such cells are available from a wide range of sources (e.g., the American Type Culture Collection, Rockland, Md.; also, see, e.g., Ausubel et al. (1994) Current Protocols in Molecular Biology, John Wiley & Sons, New York).

The methods of transformation or transfection and the choice of expression vehicle will depend on the host system selected. Transformation and transfection methods are described, e.g., in Ausubel et al. (1994) Current Protocols in Molecular Biology, John Wiley & Sons, New York; expression vehicles may be chosen from those provided, e.g., in Cloning Vectors: A Laboratory Manual (Pouwels et al., 1985, Supp. 1987).

The host cells harboring the expression vehicle can be cultured in conventional nutrient media adapted as need for activation of a chosen gene, repression of a chosen gene, selection of transformants, or amplification of a chosen gene. One expression system is the mouse 3T3 fibroblast host cell transfected with a pMAMneo expression vector (Clontech, Palo Alto, Calif.). pMAMneo provides an RSV-LTR enhancer linked to a dexamethasone-inducible MMTV-LTR promoter, an SV40 origin of replication which allows replication in mammalian systems, a selectable neomycin gene, and SV40 splicing and polyadenylation sites. DNA encoding a FasL fragment would be inserted into the pMAMneo vector in an orientation designed to allow expression. The recombinant FasL fragment would be isolated as described below. Other host cells that can be used in conjunction with the pMAMneo expression vehicle include COS cells and CHO cells (ATCC Accession Nos. CRL 1650 and CCL 61, respectively).

In mammalian host cells, a number of viral-based expression systems can be utilized. In cases where an adenovirus is used as an expression vector, the FasL fragment nucleic acid sequence can be ligated to an adenovirus transcription/translation control complex, e.g., the late promoter and tripartite leader sequence. This chimeric gene can then be inserted into the adenovirus genome by in vitro or in vivo recombination. Insertion into a non-essential region of the viral genome (e.g., region E1 or E3) will result in a recombinant virus that is viable and capable of expressing a FasL fragment gene product in infected hosts (see, e.g., Logan (1984) Proc. Natl. Acad. Sci. USA 81, 3655).

Specific initiation signals may also be required for efficient translation of inserted nucleic acid sequences. These signals include the ATG initiation codon and adjacent sequences. In cases where the FasL fragment gene or cDNA, including the native FasL gene initiation codon and adjacent sequences, is inserted into the appropriate expression vector, no additional translational control signals may be needed. In other cases, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators (Bittner et al. (1987) Methods in Enzymol. 153, 516).

In addition, a host cell may be chosen which modulates the expression of the inserted sequences, or modifies and processes the gene product in a specific, desired fashion. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include, but are not limited to, CHO, VERO, BHK, HeLa, COS, MDCK, 293, 3T3, WI38, and in particular, choroid plexus cell lines.

Alternatively, a FasL fragment can be produced by a stably-transfected mammalian cell line. A number of vectors suitable for stable transfection of mammalian cells are available to the public, see, e.g., Pouwels et al. (supra); methods for constructing such cell lines are also publicly available, e.g., in Ausubel et al. (supra). In one example, cDNA encoding the FasL fragment can be cloned into an expression vector that includes the dihydrofolate reductase (DHFR) gene. Integration of the plasmid and, therefore, the FasL fragment-encoding gene into the host cell chromosome is selected for by including 0.01-300 μM methotrexate in the cell culture medium (as described in Ausubel et al., supra). This dominant selection can be accomplished in most cell types.

A number of other selection systems can be used, including but not limited to the herpes simplex virus thymidine kinase, hypoxanthine-guanine phosphoribosyltransferase, and adenine phosphoribosyltransferase genes can be employed in tk, hgprt, or aprt cells, respectively. In addition, gpt, which confers resistance to mycophenolic acid (Mulligan et al. (1981) Proc. Natl. Acad. Sci. USA 78, 2072); neo, which confers resistance to the aminoglycoside G-418 (Colberre-Garapin et al (1981) J. Mol. Biol. 150, 1); and hygro, which confers resistance to hygromycin (Santerre et al. (1981) Gene 30, 147), can be used.

FasL fragments can be produced as fusion proteins. For example, the expression vector pUR278 (Ruther et al. (1983) EMBO J. 2, 1791), can be used to create lacZ fusion proteins. The pGEX vectors can be used to express foreign polypeptides as fusion proteins with glutathione S-transferase (GST). In general, such fusion proteins are soluble and can be easily purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In one embodiment of the present invention a fusion protein is used which comprises a FasL fragment covalently attached to a FLAG® peptide. This fusion protein can be prepared using standard recombinant techniques in which a nucleic acid encoding the FasL fragment is cloned either upstream or downstream of the coding sequence for the FLAG® peptide and expressed in an appropriate expression vector either in vitro or in vivo.

Any fusion protein can be readily purified by utilizing an antibody specific for the fusion protein being expressed. For example, a system described in Janknecht et al. (1981) Proc. Natl. Acad Sci. USA 88, 8972, allows for the ready purification of non-denatured fusion proteins expressed in human cell lines. In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns, and histidine-tagged proteins are selectively eluted with imidazole-containing buffers. Alternatively, a FasL fragment can be fused to an immunoglobulin Fc domain. Such a fusion protein can be readily purified using a protein A column.

The FasL fragments of the present invention may be fused with ligands for T cell or macrophage surface receptors, so the FasL fragments will bind to and be concentrated close to inflammatory cells, causing fatricide-mode elimination of inflammatory cells. Alternatively, the FasL fragments could be fused with ligand for receptors on CNS vascular endothelial cells, so the FasL fragments can encounter inflammatory cells as soon as they cross the blood-brain barrier. Also, the oligomerization may enhance the FasL fragment effect, so strategies such as the incorporation of a FLAG® tag are useful.

Chemical Modification of FasL Fragments

Modification of the structure of the FasL fragments can be for such purposes as enhancing therapeutic or prophylactic efficacy, stability (e.g., ex vivo shelf life and resistance to proteolytic degradation in vivo), or post-translational modifications (e.g., to alter the phosphorylation pattern of protein). Such modified fragments, when designed to retain at least one activity of the naturally occurring form of the fragments, are considered functional equivalents of the FasL fragments described in more detail herein. Such modified peptides can be produced, for instance, by amino acid substitution, deletion, or addition.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids are can be divided into four families:

(1) Acidic=aspartate, glutamate;

(2) Basic=lysine, arginine, histidine;

(3) Nonpolar=alanine, valine, leucine, isoleucine, proline, phenylalanine, methionine, tryptophan; and

(4) Uncharged polar=glycine, asparagine, glutamine, cysteine, serine, threonine, tyrosine.

In similar fashion, the amino acid repertoire can be grouped as

(1) Acidic=aspartate, glutamate;

(2) Basic=lysine, arginine, histidine;

(3) Aliphatic=glycine, alanine, valine, leucine, isoleucine, serine, threonine, with serine and threonine optionally be grouped separately as aliphatic-hydroxyl;

(4) Aromatic=phenylalanine, tyrosine, tryptophan;

(5) Amide=asparagine, glutamine; and

(6) Sulphur-containing=cysteine and methionine. (See, for example, Biochemistry, 2nd ed., Ed. by L. Stryer, W H Freeman and Co.: 1981).

Whether a change in the amino acid sequence of a peptide results in a functional homologue (e.g. functional in the sense that the resulting polypeptide mimics or antagonises the wild-type form) can be readily determined by assessing the ability of the variant peptide to produce a response in cells in a fashion similar to the wild-type FasL fragment. Polypeptides in which more than one replacement has taken place can readily be tested in the same manner.

Generally, those skilled in the art will recognize that FasL fragments as described herein may be modified by a variety of chemical techniques to produce compounds having essentially the same activity as the unmodified peptide, and optionally having other desirable properties. For example, carboxylic acid groups of the peptide, whether carboxyl-terminal or sidechain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as 5- or 6-membered. Amino groups of the peptide, whether amino-terminal or sidechain, may be in the form of a pharmaceutically-acceptable acid addition salt, such as the HCl, HBr, acetic, benzoic, toluene sulphonic, maleic, tartaric and other organic salts, or may be modified to C₁-C₁₆ alkyl or dialkyl amino or further converted to an amide. Hydroxyl groups of the peptide sidechain may be converted to C₁-C₁₆ alkoxy or to a C₁-C₁₆ ester using well-recognised techniques. Phenyl and phenolic rings of the peptide sidechain may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide sidechains can be extended to homologous C₂-C₄ alkylenes. Thiols can be protected with any one of a number of well-recognized protecting groups, such as acetamide groups.

Those skilled in the art will also recognize methods for introducing cyclic structures into the peptides of this invention to select and provide conformational constraints to the structure that result in enhanced binding and/or stability. For example, a carboxyl-terminal or amino-terminal cysteine residue can be added to the peptide, so that when oxidized the peptide will contain a disulphide bond, thereby generating a cyclic peptide. Other peptide cyclizing methods include the formation of thioethers and carboxyl- and amino-terminal amides and esters.

In addition to FasL fragments consisting only of naturally occurring amino acids, peptidomimetics or peptide analogues are also provided. Peptide analogues are commonly used in the pharmaceutical industry as non-peptide drugs with properties analogous to those of the template peptide. These types of non-peptide compound are termed “peptide mimetics” or “peptidomimetics” (Luthman, et al., A Textbook of Drug Design and Development, 14:386-406, 2nd Ed., Harwood Academic Publishers (1996); Grante (1994) Angew. Chem. Int. Ed. Engl. 33:1699-1720; Fauchere (1986) Adv. Drug Res. 15:29; Veber and Freidinger (1985) TINS, p.392; and Evans, et al. (1987) J. Med. Chem. 30:1229, which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as naturally-occurring receptor-binding polypeptide, but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods known in the art and further described in the following references: Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley (1980) Trends Pharm. Sci. pp. 463-468, (general review); Hudson, et al. (1979) Int. J. Pept. Prot. Res., 14:177-185 (—CH₂NH—, CH₂CH₂—); Spatola, et al. (1986) Life Sci., 38:1243-1249 (—CH₂—S); Hann (1982) Chem. Soc. Perkin Trans. I, 307-314 (—CH═CH—, cis and trans); Almquist, et al. (1980) J. Med. Chem., 23:1392-1398, (—COCH₂—); Jennings-White, et al, (1982) Tetrahedron Lett. 23:2533, (—COCH₂—); Szelke, et al. (1982) European Appln. EP 45665 (—CH(OH)CH₂—); Holladay, et al. (1983) Tetrahedron Lett., 24:4401-4404 (—C(OH)CH₂—); and Hruby (1982) Life Sci., 31:189-199 (—CH₂—S—); each of which is incorporated herein by reference. One example of a non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo, et al. (1992) Ann. Rev. Biochem., 61:387, incorporated herein by reference); for example, by adding internal cysteine residues capable of forming intramolecular disulphide bridges which cycles the peptide.

Synthetic or non-naturally occurring amino acids refer to amino acids which do not naturally occur in vivo but which, nevertheless, can be incorporated into the FasL fragments described herein. Exemplary synthetic amino acids are the D-α-amino acids of naturally occurring L-α-amino acid as well as non-naturally occurring D- and L-α-amino acids represented by the formula H₂NCHR⁵COOH where R⁵ is 1) a lower alkyl group, 2) a cycloalkyl group of from 3 to 7 carbon atoms, 3) a heterocycle of from 3 to 7 carbon atoms and 1 to 2 heteroatoms selected from the group consisting of oxygen, sulphur, and nitrogen, 4) an aromatic residue of from 6 to 10 carbon atoms optionally having from 1 to 3 substituents on the aromatic nucleus selected from the group consisting of hydroxyl, lower alkoxy, amino, and carboxyl, 5)-alkylene-Y where alkylene is an alkylene group of from 1 to 7 carbon atoms and Y is selected from the group consisting of (a) hydroxy, (b) amino, (c) cycloalkyl and cycloalkenyl of from 3 to 7 carbon atoms, (d) aryl of from 6 to 10 carbon atoms optionally having from 1 to 3 substituents on the aromatic nucleus selected from the group consisting of hydroxyl, lower alkoxy, amino and carboxyl, (e) heterocyclic of from 3 to 7 carbon atoms and 1 to 2 heteroatoms selected from the group consisting of oxygen, sulphur, and nitrogen, (f) —C(O)R² where R² is selected from the group consisting of hydrogen, hydroxy, lower alkyl, lower alkoxy, and —NR³R⁴ where R³ and R⁴ are independently selected from the group consisting of hydrogen and lower alkyl, (g) —S(O)ₙR⁶ where n is an integer from 1 to 2 and R⁶ is lower alkyl and with the proviso that R⁵ does not define a side chain of a naturally occurring amino acid.

Other synthetic amino acids include amino acids wherein the amino group is separated from the carboxyl group by more than one carbon atom such as β-alanine, γ-aminobutyric acid, and the like.

Testing FasL Fragments, or Derivatives Thereof, for Activity

The FasL fragments, and derivatives thereof, that are used in the methods of the present invention exhibit anti-inflammatory activity associated with their ability to induce apoptosis of activated T-cells and macrophages that express Fas.

In vitro Assays

To assay the FasL fragments, and derivatives thereof, in vitro, increasing concentrations of the candidate molecule are incubated with Fas expressing cells, e.g. or T cells or neutrophils or macrophages, and lysis of the Fas expressing cells is measured, e.g., by a ⁵¹Cr release assay. As described above, the active FasL fragments, and derivatives thereof, will have the ability to cause apoptosis of activated T cells and macrophages. Alternatively, apoptosis is measured using a TUNEL™ assay or cell viability is determined using a 3-(4,5-dimethylthiazol-2-yl)-2,5,-diphenyl tetrazolium bromide (MTT) assay. There are a variety of methods of detecting apoptosis and cell viability in vitro that are well known to workers skilled in the art, which can be used to monitor the activity of candidate FasL fragments and derivatives thereof.

Experimental Allergic Encephalomyelitis (In Vivo Assay)

Acute experimental allergic encephalomyelitis (EAE) in Lewis rats induced by MBP immunization is a well-characterized model for acute CNS autoimmune inflammation, although there is no significant CNS demyelination (Pender M. P. (1988) J Neurol Sci. 86:277-289; Wekerle H, et al (1994) Ann Neurol. 36: S47-53). Between the onset and the peak of clinical EAE, numerous T cells and macrophages cross the blood-brain barrier, accumulate in the meningeal and perivascular areas, and many also infiltrate into the CNS parenchyma, especially in the lumbosacral spinal cord. CNS inflammation parallels the EAE symptoms in both time course and severity. Moreover, the recovery from acute EAE is correlated with the apoptosis of inflammatory cells and the receding of inflammation in the CNS (Pender M. P., et al. (1992) J Autoimmun. 5:401-410).

Although immune privilege is a concept derived from transplantation studies, it represents multiple physiological mechanisms existing locally within the immune privileged site, and describes the collective effect of these mechanisms in suppressing the development of immunogenic inflammation at the site. The CNS is a relatively immune privileged site, because both the afferent and efferent limbs of a CNS-targeted immune response are suppressed by local CNS mechanisms (Keane R W. Immunosuppression: CNS effects. In: Keane R W, Hickey W F, eds. Immunology of the nervous system. New York: Oxford University Press, 1997:642-667). However, this protection is only relatively effective. Grafts with significant histocompatibility differences will be rejected from CNS (Sloan D. J. et al., (1991) Trends Neurosci. 14:341-346), and a high dose of bacillus Calmette-Guérin inoculated into brain parenchyma may also elicit CNS immunogenic inflammation (Matyszak M. K. (1998) Prog Neurobiol. 56:19-35. EAE is another example suggesting that strong autoimmune responses toward CNS antigens can overwhelm the protection derived from CNS immune privilege. Since most systemic therapies with immune modulating or suppressive effects would more or less impair the systemic physiological immune function, we are interested in exploring new strategies to suppress the CNS autoimmune inflammation through modulating the CNS microenvironment, and potentiating the CNS immune privilege.

In order to determine whether candidate molecules can inhibit the progress of EAE, rats are treated with the candidate FasL fragment, or derivative thereof, and the rats are monitored for EAE development. The degree of EAE severity is scored as follows: 0: no clinical symptoms; 0.5: incomplete tail paralysis; 1: complete tail paralysis; 2: unsteady gait, or incomplete paraplegia; 3: complete paraplegia. The active FasL fragments, and derivatives thereof, will have the ability to cause inhibit EAE progression in the rats.

Alternative in vivo models for non-autoimmune inflammation are HSV inoculation into the anterior chamber of the eye, HSV or bacillus Calmette-Guerin inoculation into the brain or spinal cord parenchyma, trauma, ischemia and reperfusion models, optic neuritis models (See, for example, Zhu B. et al. (1999) Brain Res. 824:204-217)

Use of FasL Fragments

The present invention is also directed to methods of use of the FasL fragments, or derivatives thereof, or compositions comprising a FasL fragment, or derivative thereof, for induction of apoptosis in activated T cells and macrophages and inhibition of inflammation in animals, preferably mammals, including humans. The present invention is further directed to methods of use of the FasL fragments or derivatives thereof or compositions comprising a FasL fragment or derivative thereof for the restoration or potentiation of immune privilege at immune privileged sites.

The methods of the present invention comprise administering to a subject in need thereof an effective amount of a FasL fragment, or derivative thereof, or a composition comprising a FasL fragment, or derivative thereof, to a subject to inhibit inflammation. In one embodiment, an effective amount of a therapeutic composition comprising a FasL fragment, or derivative thereof, and a pharmaceutical carrier is administered intrathecally to a subject to inhibit inflammation. In another embodiment, an effective amount of a therapeutic composition comprising a FasL fragment, or derivative thereof, and a pharmaceutical carrier is applied locally to a site to inhibit inflammation at the site.

For CNS treatment, alternative routes of administration include intraventricular, and intracisternal (through cisterna magna). Furthermore, FasL fragments can be expressed by administration of FasL fragment encoding cDNA, plasmid, liposomes, viral vectors (HSV or adenoviral), or transformed cells e.g. fibroblasts, or released by encapsulated cells, etc.

The FasL fragments, or derivatives thereof, and pharmaceutical compositions of the present invention are used in the treatment of or amelioration of inflammatory symptoms in any disease, condition or disorder where inflammation suppression would be beneficial. Inflammatory diseases, conditions or disorders in which the FasL fragments or derivatives thereof and pharmaceutical compositions of the present invention can be used to inhibit unwanted inflammation include, but are not limited to,

Further, the FasL fragments, or derivatives thereof, and compositions are also useful to treat or ameliorate inflammation associated with orchitis and epididimo-orchitis; infertility; orchidal trauma and other inflammatory-related testicular diseases, conditions or disorders where inflammation suppression would be beneficial.

In addition, the FasL fragments, or derivatives thereof, and compositions are also useful to treat or ameliorate inflammation associated with uveitis; conjunctivitis; chorioretinitis; uveoretinitis; optic neuritis; intraocular inflammation, such as retinitis and cystoid macular edema; sympathetic ophthalmia; scleritis; retinitis pigmentosa; inflammatory components of degenerative fondus disease; inflammatory components of ocular trauma; ocular inflammation caused by infection; proliferative vitreoretinopathies; acute ischemic optic neuropathy; excessive scarring, for example, following glaucoma filtration operation; inflammation reaction against ocular implants and other immune and inflammatory-related ophthalmic diseases, conditions or disorders where inflammation suppression would be beneficial.

Moreover, the FasL fragments, or derivatives thereof, and compositions are also useful to treat or ameliorate inflammation associated multiple sclerosis and other autoimmune diseases and conditions or disorders where, in the central nervous system (CNS), inflammation suppression would be beneficial; multiple sclerosis, optic neuritis;

Devic's disease; various types of encephalitis, myelitis, and encephalomyelitis: including acute disseminated encephalomyelitis, acute necrotizing hemorrhagic leukoencephalomyelitis, acute transverse myelitis, limbic encephalitis, post-polio syndrome, subacute sclerosing panencephalitis, etc; Guillian-Barre syndrome, acute, subacute, and chronic neuropathy, in which there is radiculitis within the spinal canal; aseptic meningitis, chronic and recurrent meningitis; inflammatory component of stroke, CNS trauma, CNS compression, infection, and psychiatric diseases; inflammation or rejection after CNS transplantation; inflammatory component of neurodenerative diseases including Alzheimer's disease, Parkinson's disease, Huntington's disease, amyotrophic lateral sclerosis, and HIV-related encephalopathy; other CNS diseases with autoimmune or inflammatory features: e.g. “stiff-man” syndrome.; and immune and inflammatory related diseases, conditions or disorders of the central nervous system where inflammation suppression would be beneficial.

In yet another embodiment, FasL fragments, or derivatives thereof, and compositions of the invention are useful to restore and/or potentiate immune privilege at an immune privileged site which has lost or reduced its immune privilege, such as brain, eye, placenta and testis.

An immune privileged site is defined as a site at which a graft of foreign tissue, that would be rejected promptly if placed at a conventional body site, enjoys prolonged, even indefinite, survival. The list of sites has been determined experimentally and includes the anterior chamber of the eye, the corneal stroma of the eye, the central nervous system, including the brain, the maternal-fetal interface, the adrenal cortex, the testes, the ovaries, the liver, the matrix of hair follicles, and the vitreous cavity of the eye.

In one embodiment of the present invention the FasL fragment, or derivative thereof, is administered before, after or during administration of other anti-inflammatory cytokines or peptides such as TGF-beta, IL-4, IL-10, VIP, melanocyte stimulating factor, etc. Furthermore, in the case of a FasL fragment fusion protein it can be administered with crosslinking agents, e.g. anti-FLAG® in the case of a FasL fragment-FLAG® fusion.

Gene Therapy

The FasL fragments, may also be employed in accordance with the present invention by expression of such proteins in vivo, which is often referred to as “gene therapy.”

Thus, for example, cells from a patient may be engineered with a polynucleotide (DNA or RNA) encoding a polypeptide ex vivo, with the engineered cells then being provided to a patient to be treated with the polypeptide. Such methods are well-known in the art. For example, cells may be engineered by procedures known in the art by use of a retroviral particle containing RNA encoding a FasL fragment according to the present invention.

Similarly, cells may be engineered in vivo for expression of a polypeptide in vivo by, for example, procedures known in the art. As known in the art, a producer cell for producing a retroviral particle containing RNA encoding FasL fragment according to the present invention, may be administered to a patient for engineering cells in vivo and expression of the polypeptide in vivo. These and other methods for administering FasL fragment according to the present invention, by such method should be apparent to those skilled in the art from the teachings of the present invention. For example, the expression vehicle for engineering cells may be other than a retrovirus, for example, an adenovirus which may be used to engineer cells in vivo after combination with a suitable delivery vehicle.

Retroviruses, from which the retroviral plasmid vectors hereinabove mentioned, may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumour virus. In one embodiment, the retroviral plasmid vector is derived from Moloney Murine Leukemia Virus.

The vector includes one or more promoters. Suitable promoters which may be employed include, but are not limited to, the retroviral LTR; the SV40 promoter; and the human cytomegalovirus (CMV) promoter described in Miller, et al., Biotechniques, Vol. 7, No. 9, 980-990 (1989), or any other promoter (e.g., cellular promoters such as eukaryotic cellular promoters including, but not limited to, the histone, pol III, and β-actin promoters). Other viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The nucleic acid sequence encoding the polypeptide of the present invention is under the control of a suitable promoter. Suitable promoters which may be employed include, but are not limited to, adenoviral promoters, such as the adenoviral major late promoter; or hetorologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAl promoter; human globin promoters; viral thymidine kinase promoters, such as the Herpes Simplex thymidine kinase promoter; retroviral LTRs (including the modified retroviral LTRs hereinabove described); the β-actin promoter; and human growth hormone promoters. The promoter also may be the native promoter which controls the genes encoding the polypeptides.

The retroviral plasmid vector is employed to transduce packaging cell lines to form producer cell lines. Examples of packaging cells which may be transfected include, but are not limited to, the PE501, PA317, ψ-2, ψ-AM, PA12, T19-14X, VT-19-17-H2, ψCRE, ψCRIP, GP+E-86, GP+envAm12, and DAN cell lines as described in Miller, Human Gene Therapy, Vol. 1, pgs. 5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or PTH to a lipid, and then administered to a host.

The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

Pharmaceutical Formulations

The present invention is also directed to therapeutic or pharmaceutical compositions comprising a pharmaceutically acceptable carrier and an anti-inflammatory FasL fragment or derivative thereof In an exemplary embodiment, the composition contains recombinant FasL comprising amino acids 103-281 of the full length FasL as the active ingredient.

The amount of the therapeutic or pharmaceutical composition of the invention which is effective in the treatment of a particular disease, condition or disorder will depend on the nature of the disease, condition or disorder and can be determined by standard clinical techniques. In vitro assays may optionally be employed to help identify optimal dosage ranges. The precise dose to be employed in the formulation will also depend on the seriousness of the disease, condition or disorder, and should be decided according to the judgment of the practitioner and each patient's circumstances. Effective doses may be extrapolated from dose-response curves derived from in vitro or animal model test systems.

The invention also provides a pharmaceutical pack or kit comprising one or more containers filled with one or more of the ingredients, i.e., peptide, carrier, of the pharmaceutical compositions of the invention.

### EXAMPLES

Methods and Methods

EAE Induction And Observation

Male Lewis rats with a body weight between 175 g and 200 g were obtained from Charles River Animal Laboratory, Canada. The protocols for animal experiments were approved by the Animal Care Center, University of British Columbia. For the actively induced EAE in Lewis rats, each rat was immunized subcutaneously on both sides of the abdominal flank close to the inguinal lymph nodes with a total of 100 μl myelin basic protein (MBP)/complete Freud's adjuvant (CFA) emulsion, which contained 50 μg guinea pig MBP (Sigma) and 500 μg heat-inactivated mycobacteria tuberculosis (Difco). For the passively transferred EAE, 1-2×10⁶ MBP-specific T line cells were injected intravenously into each Lewis rat under anesthesia. The rats were weighed and scored for EAE severity daily over 20 days post immunization (dpi) or 15 days after adoptive transfer. The degree of EAE severity was scored as follows: 0: no clinical symptoms; 0.5: incomplete tail paralysis; 1: complete tail paralysis; 2: unsteady gait, or incomplete paraplegia; 3: complete paraplegia.

Intrathecal Infusion in Lewis Rats

The 14-day osmotic minipumps (Alza) were each filled with 200 μl of artificial cerebrospinal fluid (CSF) (Kehne J. H. et al. (1986) J Neurosci. 6:3250-3257) supplemented with 2 μl of 10 mg/ml gentamycin. The pumps were connected to a 6.5 cm PE-10 tube (Intramedic) through a 3 cm plastic tube and an infusion switch that are included in the brain infusion kit (Alza). The assembled minipumps were immersed in saline and left in a 37° C. tissue culture incubator overnight. To implant minipumps, the subarachnoid matter covering the cisterna magna was exposed by dissection. After a small opening was made in the subarachnoid matter, the 6.5 cm PE-10 tube was inserted into the subarachnoid space. The tip of the PE-10 tube was close to T12 of the rat spinal cord. The minipumps were embedded subcutaneously between the scapulae. Most of the rats started to regain their body weight 3 to 4 days after the surgery. On the sixth day after the surgery, the rats were immunized with MBP/CFA as described above. At 7 dpi, the original 14-day minipumps were changed to 3-day minipumps to infuse rFasL (Upstate Biotechnology catalog #01-193) or the control solution between 7 dpi and 10 dpi. The rFasL used in the present example is a recombinant fusion protein corresponding to the whole extracellular domain of human Fas ligand (amino acids 103-281 inclusive; GenBank accession #I38707), covalently linked to a FLAG® domain.

For rFasL infusion, the 100 μl solution contained 125 ng to 700 ng of rFasL, 20 μl of 7.05 TIU/ml aprotinin, 1 μl of 10 mg/ml gentamycin in the artificial CSF. Aprotinin, a protease inhibitor, was used to prevent the degradation of rFasL in the inflammatory environment. For control infusion, the 100 μl solution was the same but only without rFasL. These filled 3-day pumps were immersed in saline and left in a 37° C. tissue culture incubator for 5 hours before use. Under anesthesia, a small incision was made between the two scapulae, and the 14-day pumps were replaced with the 3-day pumps.

Morphological Techniques

The Lewis rats were sacrificed by intraperitoneal Euthanyl injection, and immediately perfused transcardially with 300 ml PBS. The lumbosacral spinal cord (LSSC) was dissected out and cut into 9 segments corresponding to the L1 to S3 spinal cord segments. These segments were immersed in TissueTek in a cryomold and were oriented with the rostral ends towards the bottom of the wells. They were then frozen in liquid nitrogen-cooled 2-methylbutane for 2 minutes, and stored in a −80° C. freezer until use. The 10 μm frozen tissue sections were cut on a Reichert-Jung 2800 Frigocut cryostat, and the slides were stored in a −20° C. freezer for less than a month before staining. The Luxol fast blue staining, Toluidine blue staining and immunostaining were performed following the previous protocols (Zhu B. et al. (1999) Brain Res. 824:204-217). TUNEL staining was performed according to the protocol supplied by Oncogene.

Quantitation of CNS Inflammation

A graticulate with 0.5 mm grids was mounted onto the eyepiece of a Nikon Optiphot-2 microscope. Inflammation was quantified by two methods. One involved counting the inflammatory foci in the rat LSSC parenchyma. An inflammatory focus was defined as the presence of a cluster of 20 or more aggregated mononuclear cells (Sobel R. A. et al. (1984) J Immunol. 132:2393-2401). The ‘count of inflammatory foci’ represents the total number of inflammatory foci in 9 HE-stained LSSC sections from each different level. Another method used was the ‘score of inflammation’. We selected the ventral-medial white matter as a standard area for comparison. Under 400×magnification (the diameter of the viewfield was 0.375 mm), the viewfield was focused on one side of the most medial ventral white matter area without including the meninges and the anterior medium fissure. The numbers of total cell nuclei were counted on both sides of the 9 different level HE-stained LSSC sections (a total of 18 viewfields for each animal), and the average for one viewfield was calculated. The score of inflammation for a particular spinal cord was the difference between that average and the average from 3 normal rat spinal cords. Essentially, the score of inflammation reflects the increase in cell numbers in the ventral-medial LSSC white matter. Because we did not observe any neural cell proliferation in the LSSC of EAE rats, the increase in cell number reflects the degree of inflammation. Positively immunostained cells were quantitated in the same 18 medial ventral white matter viewfields, and the average was calculated. It reflected the average number of immunopositive cells within a single viewfield.

Cell Culture

MBP-specific T cell lines were established according to the standard protocol. Briefly, the monocytes from the draining lymph nodes of MBP/CFA immunized Lewis rats were isolated on 9 dpi, and stimulated in vitro for 72 hrs with 20 ug/ml MBP. The viable lymphoblasts were purified by Ficoll density centrifugation, and expanded with 4 ng/ml IL-2 (Sigma) for five days. Cells were maintained through additional cycles of MBP stimulation in the presence of gamma-irradiated syngenic thymocytes, and expansion induced by IL-2. Usually 1×10⁶ T line cells are sufficient to transfer 3⁰EAE after two rounds of MBP-stimulation in vitro.

Inflammatory peritoneal macrophages were obtained according to the reported protocols (Naraba H. et al. (1998) J Immunol 160:2974-2982). Briefly, 5% proteose peptone (Sigma) in saline (5 ml/100 g body weight) was injected intraperitoneally into Lewis rats. The cells in peritoneal exudates were collected after 72 hrs by washing the peritoneal cavity with 20 ml of ice-cold Ca²⁺ and Mg²⁺-free HBSS. The cells were seeded into 96-well plates at 1.2×10⁶ cells/well in RPMI-1640 medium containing 10% FBS. After 2 hrs in a tissue culture incubator, nonadherent cells were removed by rinsing. Then RPMI-1640 medium containing 10% FBS was added to the adherent cells, and over 90% of these cells were identified as macrophages by Giemsa staining.

T Cell Proliferation

T cell proliferation assays were performed first by obtaining the inguinal lymph nodes in immunized rats on either 10 dpi or 12 dpi, corresponding to the EAE onset and EAE peak in control rats respectively. A single cell suspension of the inguinal lymph nodes was prepared, and the red blood cells and dead cells were removed by histopaque- 1077 (Sigma) gradient centrifugation. Cells were inoculated into 96-well plates at 4×10⁵/well, and cultured in RPMI-1640 medium (Gibco) containing 5% fetal bovine serum, 2 mM L-glutamine, 50 mM 2-mercaptoethanol and 20 μg/ml MEBP. 72 hours later, 0.5 μCi [³H]-thymidine was added into each well. After 16 hours of incubation, the cells were harvested and the radioactivity was read on a scintillation counter. In some experiments, T cell proliferation assay was also performed using MBP-specific T line cells. After expansion in IL-2, 8×10⁴ T blasts per well were cultured with 3×10⁵ gamma-irradiated syngenic thymocytes and 10 ug/ml MBP in the same medium as described above. 48 hours later, 0.5 μCi [³H]-thymidine was added into each well. After 16 hours of incubation, the cells were harvested and the radioactivity was read on a scintillation counter.

Delayed-Type Hypersensitivity

The delayed-type hypersensitivity (DTH) tests were performed on 12 dpi. 50 μl of 0.75 μg/μl MBP solution was injected intradermally at the dorsal aspect of the rat right ear. The thickness of the right ear was measured 5 times both before injection and 24 hours after injection. The average increase in ear thickness after injection was recorded as the DTH response to MBP for that specific rat. The injection of 50 μl of saline solution as controls did not result in any thickness increase over 24 hours.

MTT Assay

A solution of 5 mg/ml 3-(4,5-dimethylthiazol-2-yl)-2,5,-diphenyl tetrazolium bromide (MTT) in PBS was added to macrophages at 1:10 dilution. After incubation at 37° C. for 3 hrs, the cells were rinsed with PBS twice, and the purple crystals were dissolved in 200 ul of isopropanol. The cell debris was removed by centrifugation at 14,000 rpm×5 min, and the supernatant was transferred into a new 96-well plate. MTT results represent the differences in absorption between 560 nm and 690 nm read on a microplate reader.

Annexin V-FITC/PI Staining and Flow Cytometry

Annexin V-FITC/PI staining was performed according to the PharMingen protocol. Briefly, 5 ul of Annexin V-FITC and 10 ul of 50 ug/ml PI were added to 100 μl of cell suspension in 1×binding buffer (10 mM HEPES/NaOH, pH7.4, 140 mM NaCl, 2.5 mM CaCl₂). After 15 min incubation in the dark, 400 ul of 1×binding buffer was added to each sample, and the samples were analyzed by flow cytometry within one hour.

Collection of Rat CSF

After the Lewis rats were anesthetized, the subarachnoid matter covering the cisterna magna was exposed by dissection. The tip of a 30-gauge needle attached to a syringe was inserted into the subarachnoid space, and about 150-200 ul clear CSF was collected. The albumin levels in pooled CSF were lower than 1/1000 of rat serum albumin level, suggesting there was no blood contamination in the collected rat CSF.

Statistics

Two-sample t test was employed to compare mean values between two groups. Values of p<0.05 were regarded as statistically significant.

### Example I

### Clinical Signs of EAE

A summary of the clinical effects of rFasL treatment in acute EAE is shown in Table 1. Since the pain and stress might interfere with the EAE development in experimental animals (Kuroda Y. et al. (1994) Brain Res Bull. 34:15-17) due to the surgical procedures, the incidence of clinical EAE, the EAE onset, the peak EAE score and the loss of body weight during EAE between control-infused rats and non-infused rats were comparied. These two groups did not differ significantly among any of the above four measures. This indicates that the procedure of intrathecal infusion as well as the ingredients in the control infusion solution did not interfere with the development of acute EAE. In rFasL treatment experiments, fifteen rats were each infused with 350 ng rFasL during 7˜10 dpi. (MBP-immunized rats typically developed EAE on 10 dpi or 11 dpi.) It was found that clinical EAE was completely prevented in 12 rats (80%). In three other rats that developed EAE symptoms, the EAE onset was significantly delayed (12.3±0.3 vs. 10.6±0.2, p<0.001), and the EAE severity (0.8±0.1 vs. 2.9±0.2, p<0.001) and weight loss (22.3±6.1 vs. 44.5±2.1, p<0.001) were also significantly reduced.

The effects of rFasL infusion at doses of 175 ng and 700 ng per rat were also compared. Among the eight rats that were infused with 175 ng of rFasL, five developed clinical EAE. The EAE onset was also delayed (12.2±0.4 vs. 10.6±0.2, p<0.001), and EAE severity (1.3±0.3 vs. 2.9±0.2, p<0.001) and the weight loss (28.0±4.9 vs. 44.5±2.1, p<0.00 1) were reduced. In contrast, when ten Lewis rats were intrathecally infused with 700 ng rFasL, none of them developed any symptoms of EAE over twenty days after immunization. They moved actively, and their body weight increased at a rate similar to that of the normal rats. Taken together, these data suggest that intrathecal infusion of rFasL dose-dependently suppressed acute EAE in Lewis rats.

### Example II

### Neuroimmunopathology

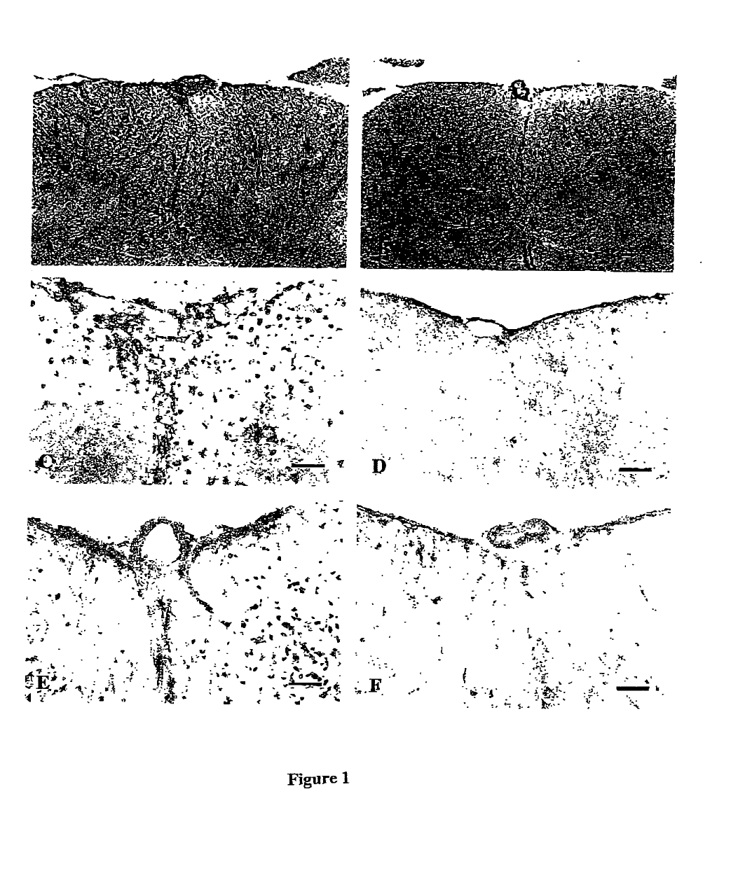

Since inflammation is most severe in the LSSC in this EAE model (Simmons R. D. et al. (1992) Autoimmunity 14:17-21; Matsuda M. et al. (1994) Autoimmunity. 19:15-22), the degree of inflammation in LSSC was compared between four control-infused rats (all with 3⁰ EAE) and four rats infused with 350 ng of rFasL (three were EAE-free, and one with 1⁰ EAE). The tissues were all obtained on 12 dpi. HE staining (FIG. 1A) shows that control-infused rats at the peak EAE stage developed severe inflammation in the LSSC, most significantly in the meningeal and perivascular areas, but many inflammatory cells also infiltrated into the parenchyma of the spinal cord. In contrast, minimal inflammation was observed in LSSC of rFasL-infused rats (FIG. 1B). In the rat that was infused with 350 ng of rFasL and developed mild EAE, the degree of inflammation in the LSSC was also found to be much milder. Previous studies have suggested that T lymphocytes and macrophages represent most of the inflammatory cells in EAE (Raine C S. (1984) Lab Invest. 50:608-635). To compare the presence of these two cell populations in LSSC sections between the control infusion group and the rFasL infusion group, immunostaining was performed with multiple markers. ED1 staining is a marker for macrophages, but also weakly stains the granulocytes. OX19 (anti-rat CD5) and W3/13 (anti-rat CD43) are markers mainly for the T cells, but also stain some B cells and polymorphonuclear cells respectively. OX42 is a marker for microglia and infiltrated macrophages, and W3/25 is a marker for CD4+ cells. All of these markers showed significant decreases following 350 ng rFasL infusion (typical ED1 and OX19 staining are shown in FIGS. 1C, D, E, F).

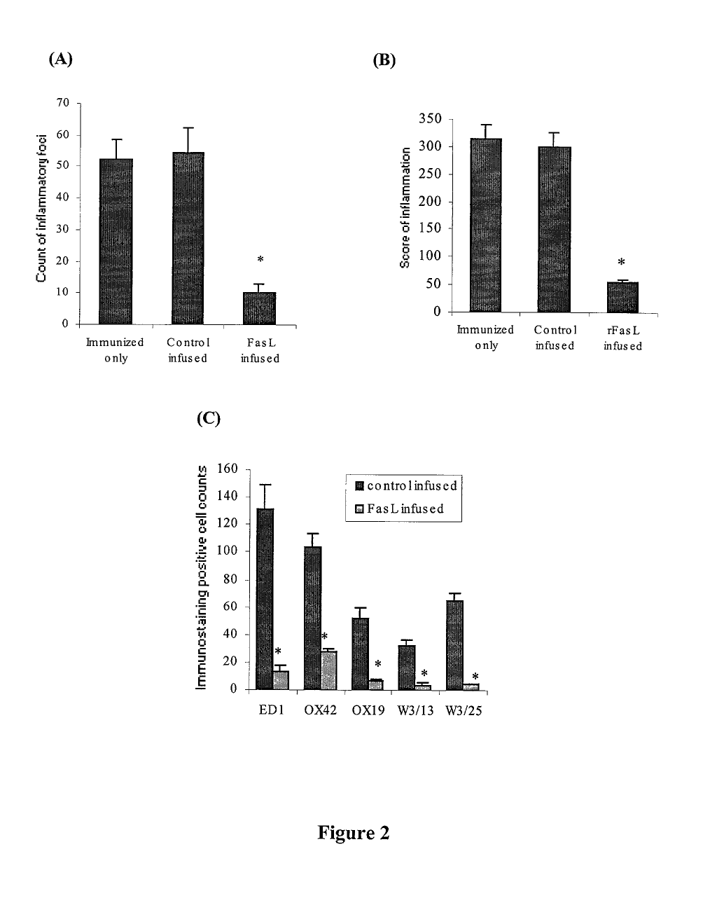

To quantitate the degrees of inflammation in LSSC, two pathological indices were compared, i.e. the count of inflammatory foci and the score of inflammation, in three groups of animals: non-infused EAE rats (n=3, EAE 3⁰), control-infused EAE rats (n=4, EAE 3⁰), 350 ng rFasL-infused rats (n=4, three were EAE-free and one with 1⁰ EAE). (FIG. 2) RFasL infusion significantly reduced the count of inflammatory foci by 80% and the score of inflammation by 83%, but control infusion had no significant effect on both parameters. This suggests that while control infusion did not affect LSSC inflammation, rFasL infusion greatly inhibited spinal cord inflammation in this EAE model. The cells that were positive in ED1, OX42, OX19, W3/13, and W3/25 immunostaining were quantitated and it was found that rFasL infusion decreased positively stained cells by 91%, 73%, 89%, 91% and 94% respectively. (FIG. 2C) The less reduction in OX42 positive cells could be due to the inclusion of some OX42+ resident microglia (Spanaus K. S. et al (1998) Eur J ImmunoL 28:4398-4408). Taken together, the data indicate that 350 ng rFasL infusion greatly reduced the infiltration of T cells and macrophages in the rat LSSC.

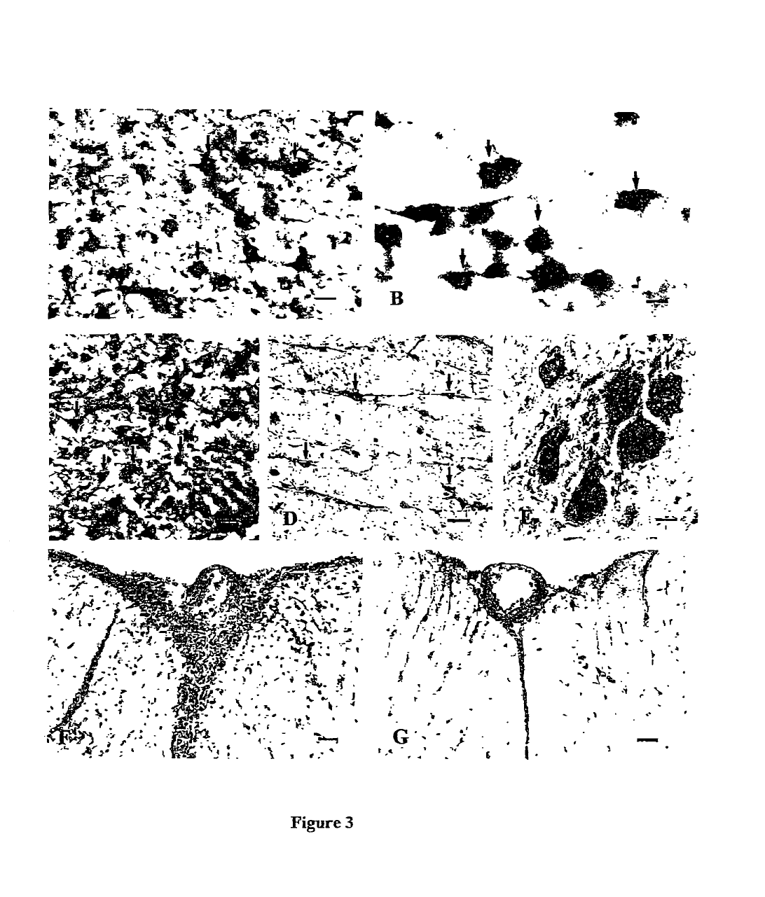

Studies were performed to determine 1) whether infiltrated T cells and macrophages in the EAE spinal cord express Fas receptors, 2) whether neurons, astrocytes or oligodendrocytes in the normal spinal cord express Fas receptors, and 3) how the pattern of Fas+ cells was changed after rFasL infusion. Double immunolabeling clearly showed that Fas receptors were expressed on both OX19+ cells and ED1+ cells, suggesting that these inflammatory cells could be the targets for rFasL (FIG. 3A, B). Anti-GFAP, Rip and SMI-32 monoclonal antibodies were specific markers for astrocytes, oligodendrocytes and neurons respectively. Double labeling of Fas on GFAP, Rip, and SMI-32 immunostaining positive cells in the normal rat spinal cord was observed, suggesting Fas receptors were constitutively expressed on astrocytes, oligodendrocytes, and neurons in the rat spinal cord. (FIGS. 3C, D, E) When Fas immunostaining was compared between control-infused and rFasL-infused rat LSSC sections, rFasL infusion was found to dramatically decrease the incidence of Fas+ cells in LSSC. (FIGS. 3F, G) The remaining Fas+ cells after rFasL infusion exhibited the same pattern of Fas immunostaining as in the normal rat LSSC sections (data not shown), suggesting that most of them were Fas+ spinal cord neural cells, which were not affected by the rFasL infusion.

### Example III

### In vitro rFasL Effects on Activated T Cells and Macrophages

Although activated T cells (Dhein J. et al (1995) Nature 373:438-441; Brunner T. et al. (1995) Nature 373:441-444; Ju S. T. et al. (1995) Nature 373:444-448), B cells (Watanabe D. et al (1995) Int Immunol. 7:1949-1956), macrophages (Ashany D. et al. (1995) Proc Natl Acad Sci USA. 92:11225-11229; Kiener P. A. et al. (1997) J Immunol 159:1594-1598, and granulocytes (Brown S. B. et al. (1999) J Immunol. 162:480-485) were observed to be susceptible to different forms of FasL in several experimental models, the in vitro effects of rFasL that contains the entire extracellular domain of FasL on inflammatory cells closely related to EAE, i.e. encephalitogenic T cells and activated macrophages have not been determined.

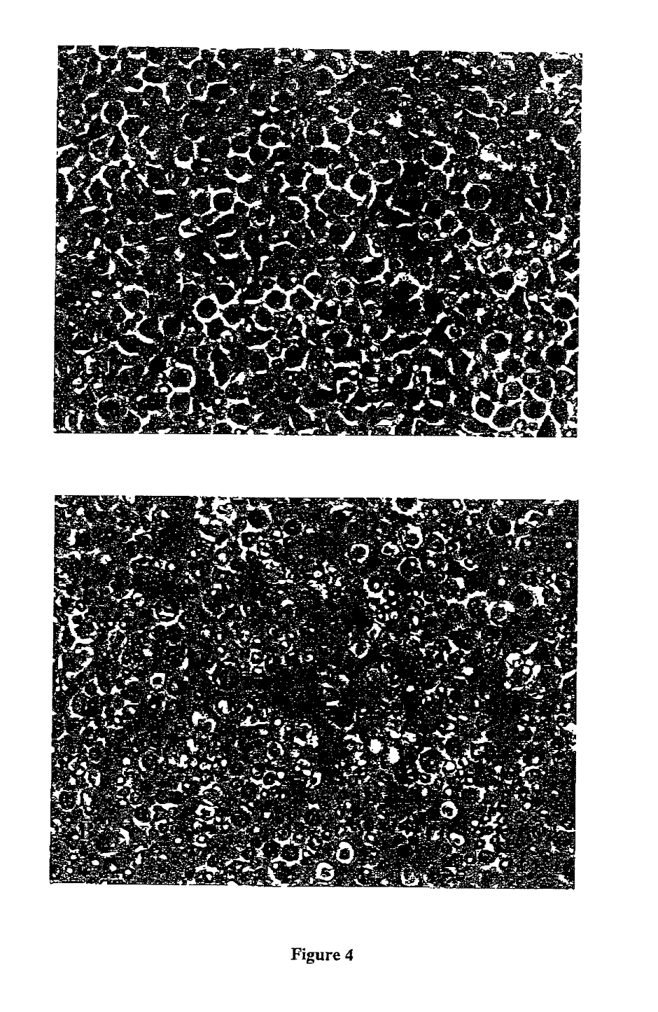

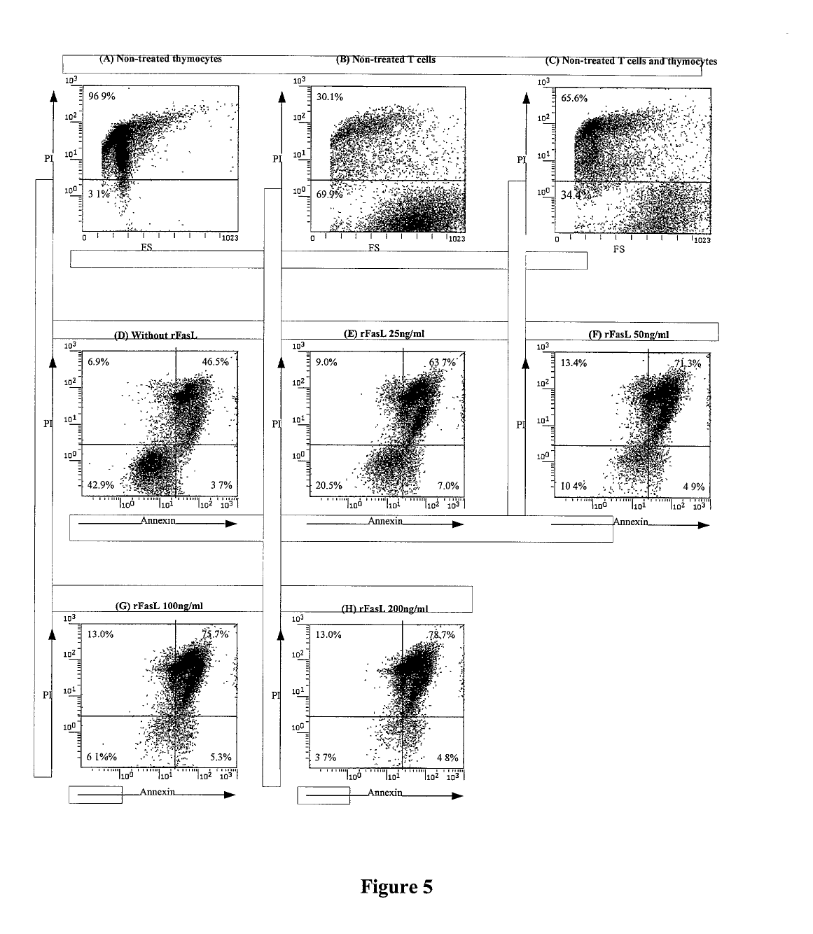

First, T cells lines from the draining lymph nodes of MBP-immunized Lewis rats were established. After the second round of in vitro MBP stimulation, 1×10⁶ T blasts were sufficient to transfer 3⁰ EAE (complete hind limb paralysis) in the recipient naive rats. RFasL was tested on these T cells. As shown in FIG. 4A, non-treated T line cells were almost confluent in culture, and they were much bigger than co-cultured gamma-irradiated thymocytes. After 16 hrs of treatment with 200 ng/ml rFasL, very few T blasts were still alive. (FIG. 4B) The great susceptibility of these T blasts to rFasL was further shown in Annexin V-FITC/propidium iodide (PI) staining analyzed by flow cytometry. (FIG. 5) Because virtually all gamma-irradiated thymocytes were positive in PI staining after two days in culture (FIG. 5A), most PI-negative cells in co-culture were the bigger T blasts (FIGS. 5B, 5C). With the doses of rFasL from 0 to 25 ng/ml, 50 ng/ml, 100 ng/ml and 200 ng/ml, T blasts double negative for Annexin V-FITC and PI staining among total cells decreased significantly from 42.9% to 20.5%, 10.5%, 6.1% and 3.7% respectively. (FIGS. 5D to 5H) Over 90% of T blasts were killed by 200 ng/ml rFasL treatment within 16 hrs.

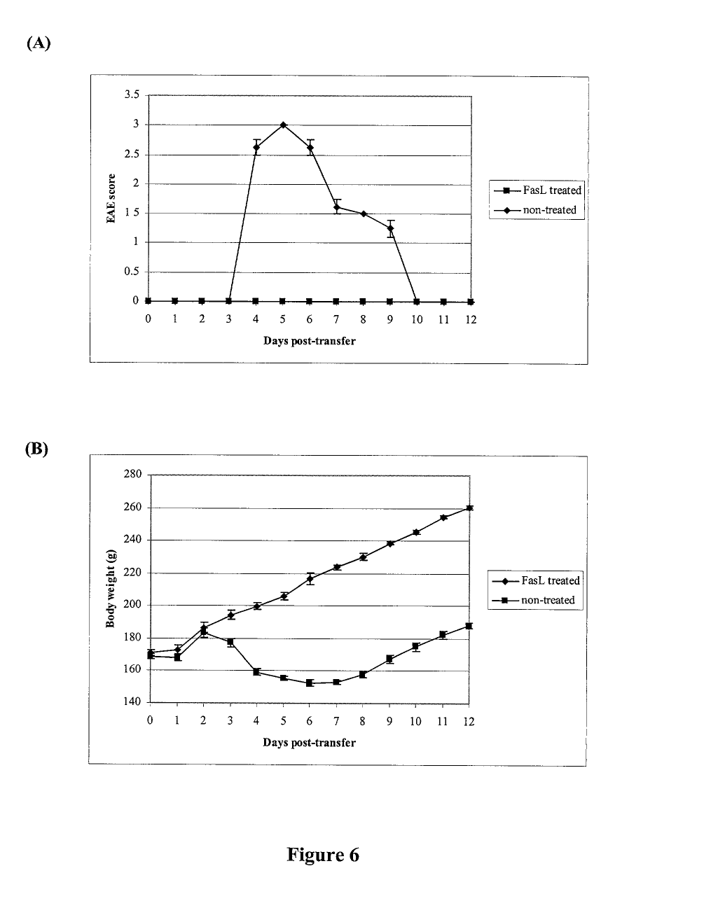

Next, the changes in encephalitogenicity of T line cells after rFasL treatment were examined in an adoptive transfer EAE model. Although 1×10⁶ T blasts were sufficient to transfer 3⁰ EAE, 2×10⁶ T blasts were transferred into each of the four naive rats, and the cells from duplicate culture but treated with 200 ng/ml rFasL for 16 hours were transferred into each of other four naive rats. (Controlled by same cell collection and transferring procedures) As shown in FIG. 6, four rats transferred with non-treated T blasts all developed 3⁰EAE, and the average body weight decrease during the EAE course was over 30 grams. In contrast, none of those four rats transferred with rFasL treated cells developed any symptom of EAE, or had any decrease in their body weights. These data show that rFasL treatment is able to completely abrogate the encephalitogenicity of MBP-specific T line cells within 16 hours.

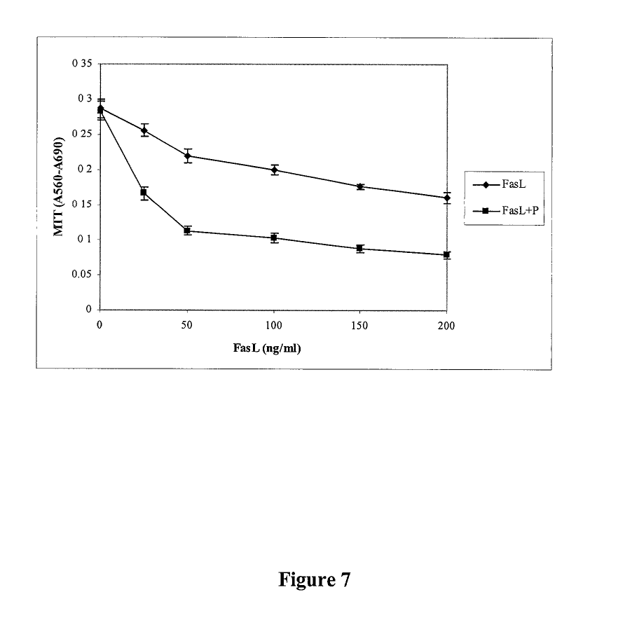

In order to determine whether activated macrophages were also susceptible to rFasL-mediated killing, peritoneal inflammatory macrophages were first activated with 100 U/ml IFN-gamma for 24 hours, and then triggered with lipopolysaccharide (LPS). A dose-dependent effect in killing macrophages was observed with rFasL treatment for 16 hrs. (FIG. 7) Up to 45% of macrophages were killed by 200 ng/ml rFasL treatment. When an anti-FLAG® antibody (1.5 μg/ml) which crosslinks rFasL by the FLAG® tail was added, enhanced killing effects were observed, and over 70% of activated macrophages were eliminated with 200 ng/ml rFasL. These results suggest that while activated macrophages are not as sensitive to rFasL as activated T line cells, majority of activated macrophages are susceptible to FasL-induced cell death, depending on the format of FasL that is administered.

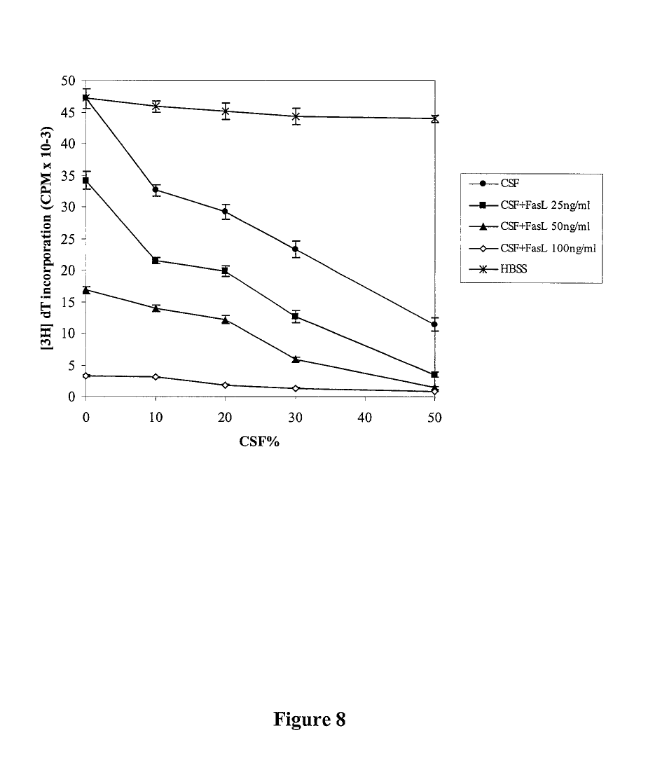

The effects of rat CSF alone or together with different doses of rFasL on MBP-induced proliferation in MBP-specific T line cells was also examined. (FIG. 8) Different percentages (V/V) of CSF, or HBSS (as controls) were included from the beginning of T cell proliferation experiment. While 10%-50% HBSS had no significant effect on T cell proliferation, the inclusion of 10%-50% CSF dose-dependently inhibited T cell proliferation. The inclusion of 50% CSF inhibited 76% of T cell proliferation. This inhibitory effect was further enhanced dose-dependently by rFasL treatment. With 50% CSF in culture, any tested dose of rFasL reduced T cell proliferation by over 90%. These data suggest that while CSF has a profound immunosuppressive function, exogenous FasL greatly potentiate this suppression.

Collectively, above in vitro data showed the strong apoptosis-inducing property of rFasL in activated MBP-specific T line cells and activated macrophages, and the synergistic immunosuppressive function between rFasL and CSF.

### Example IV

### Potentiation of CNS Immune Privilege

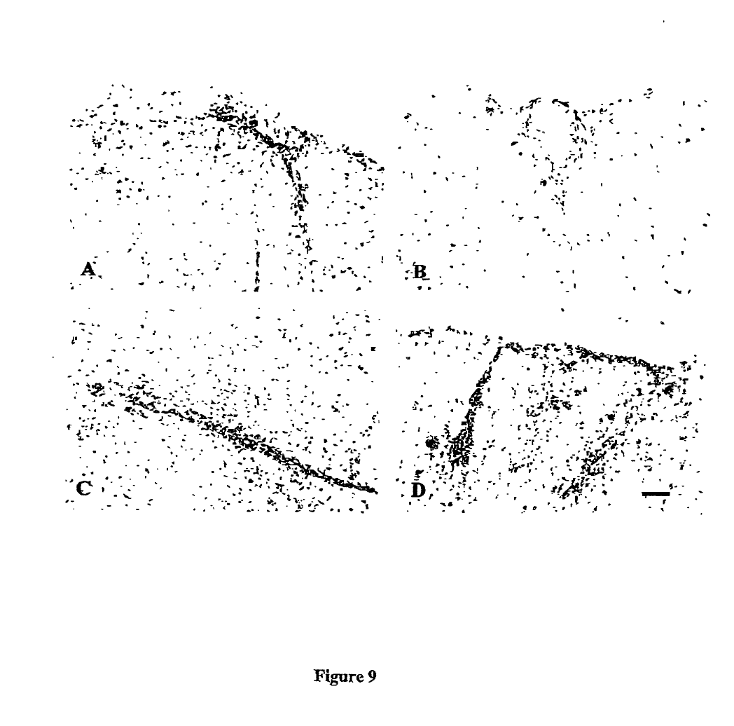

To study the in vivo mechanism of EAE suppression by rFasL infusion, the TUNEL staining patterns were compared between control-infused and rFasL-infused animals. When control-infused rats (n=3) reached their EAE peak, TUNEL+ cells appeared mostly in the parenchyma of LSSC (FIG. 4A), but were rare in the meningeal and perivascular areas (FIGS. 9A, C). In rFasL-infused rats (n=3) that had no EAE symptoms on 12 dpi, the TUNEL+ cells were less common in both the parenchyma and meningeal/perivascular areas. (FIG. 9B) The same situation was observed when TUNEL staining was performed on LSSC sections obtained on 10 dpi from rFasL-infused and EAE free rats (n=3). This was expected since the inflammation was minimal in LSCC of rFasL-infused and EAE free rats at all time points examined, i.e. on 9 dpi, 10 dpi, 12 dpi and 15 dpi. (Some data not shown) This suggested that the total number of inflammatory cells entering the CNS after rFasL infusion was much less than that in controls. In the development of EAE, only small numbers of activated antigen-specific T cells first cross the blood-tissue (in this case the blood-brain) barrier and enter the CNS perivascular areas. With the pro-inflammatory feedback from these cells, a much larger second-wave infiltration of both T cells and macrophages leads to the severe CNS inflammation and the initiation of EAE disease (Wekerle H, et al. (1994) Ann Neurol. 36:S47-53).

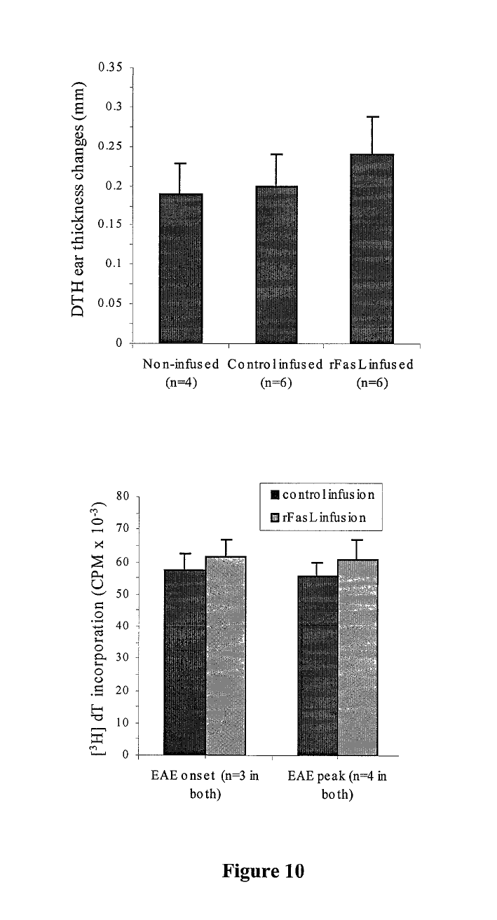

To determine whether the suppression of acute EAE after intrathecal rFasL infusion might also contributed by the suppression of the systemic immune response to MBP, the MBP-induced DTH response on 12 dpi, and MBP-induced T cell proliferation on both 10 dpi and 12 dpi between control-infused and 350 ng rFasL-infused, EAE-free animals were compared. As shown in FIG. 10, neither the DTH responses nor the T cell proliferation to MBP differed significantly between the two groups. These results exclude the possibility that systemic immune deviation or tolerance to MBP played a role in suppressing the acute EAE after rFasL infusion.

To further exclude the possibility that intrathecally infused rFasL might be drained with CSF outside the CNS and exert its action on the systemic immune system, intramuscular or intravenous injections of similar doses of rFasL were tested to determine whether they could prevent acute EAE in Lewis rats. When 175 ng of rFasL were injected intramuscularly (n=9) or intravenously (n=4) twice daily on both 8 dpi and 9 dpi, no significant changes in the EAE onset, EAE severity, or the weight loss during EAE were observed. (Table 1)

Combining the results from above three experiments, it is demonstrated that rFasL prevents EAE by creating a barrier at the perivascular space, where the infiltrating inflammatory cells are eliminated before they would enter the spinal cord parenchyma.

No Damage On Neural Cells or Myelin

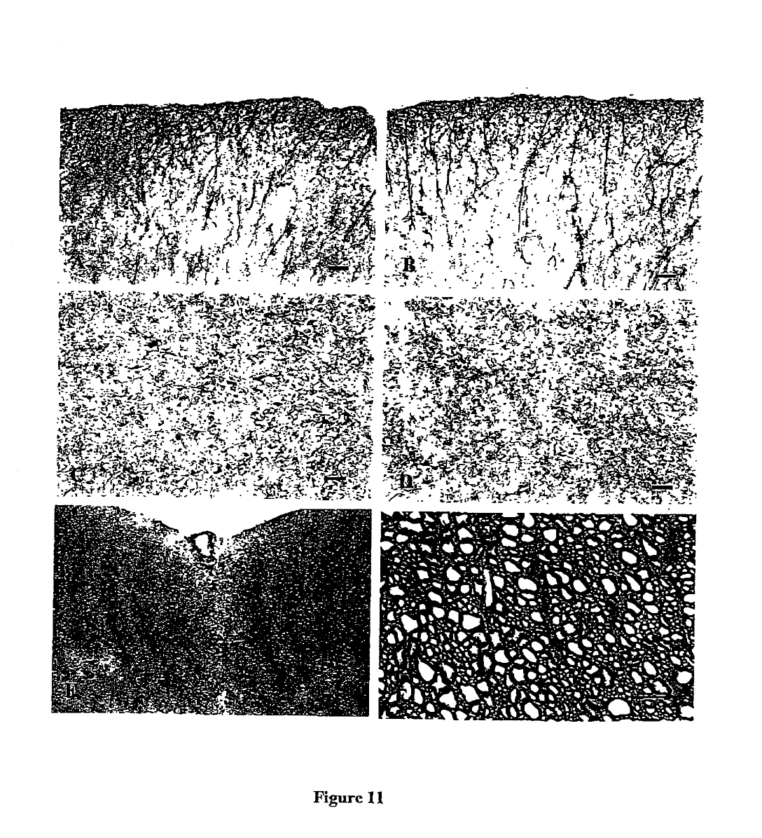

As mentioned above, neurons, astrocytes, and oligodendrocytes in the rat spinal cord constitutively were found to express Fas receptors. This raised the possibility that rFasL infusion might cause cytotoxicity in normal neural cells, although the patterns of Fas+ cells in LSSC were normal after rFasL infusion. Immunostaining with anti-GFAP, Rip and SMI-32 antibodies on 700 ng rFasL-infused and normal rat LSSC sections was compared. No changes were observed in either the densities or the morphology of these neural cells (FIGS. 11A-D, neuronal staining not shown). Luxol fast blue staining on 700 ng rFasL-infused LSSC sections, demonstrated no white matter areas with myelin loss (FIG. 11E). Toluidine blue staining was also performed on semi-thin LSSC sections from 700 ng rFasL infused rats. It showed that the myelin sheaths were intact and the myelin thickness was normal (FIG. 11F). In conclusion, no toxic effects on the neural cells or the myelin structure in LSSC was observed after infusion of 700 ng rFasL.

TABLE 1
Intrathecal infusion but not systemic application of rFasL prevents acute
EAE in Lewis rats

| | | | EAE onset (days | | |
|---|---|---|---|---|---|
| Experimental | Number of | Incidence of | post- | Peak EAE | Loss of body |
| groups | rats | clinical EAE | immunization | score | weight (g) |
| No infusion | 10 | 100% (10/10) | 10.5 ± 0.3 | 2.8 ± 0.2 | 42.1 ± 2.5 |
| Control infusion | 15 | 100% (15/15) | 10.6 ± 0.2 | 2.9 ± 0.2 | 44.5 ± 2.1 |
| rFasL intrathecal | 8 | 63% (5/8) | 12.2 ± 0.4* | 1.3 ± 0.3* | 28.0 ± 4.9* |
| infusion (175 ng) | | | | | |
| rFasL intrathecal | 15 | 20% (3/15) | 12.3 ± 0.3* | 0.8 ± 0.1* | 22.3 ± 6.1* |
| infusion | | | | | |
| (350 ng) | | | | | |
| rFasL intrathecal | 10 | 0% (0/10) | N/A | N/A | N/A |
| infusion (700 ng) | | | | | |
| rFasL injections | 9 | 100% (9/9) | 10.1 ± 0.5 | 2.7 ± 0.2 | 40.4 ± 4.1 |
| i.m. (175 ng × 4) | | | | | |
| rFasL injections | 4 | 100% (4/4) | 10.7 ± 0.5 | 2.8 ± 0.3 | 47.0 ± 7.1 |
| i.v. (175 ng × 4) | | | | | |

1) The rats in all experimental groups were immunized with MBP/CFA., and observed over 20 dpi.
2) Data regarding EAE onset, EAE severity and loss of body weight present mean ± SEM from rats that developed clinical EAE.
3) The differences in values between rFasL-treated groups and the control-infused group were examined for statistical significance by a student t test. (* P<0.05)

We claim:

1. A method of modulating inflammation within an immune privileged site in an animal by introducing an effective amount of a Fas ligand fragment comprising the extracellular domain of a full length Fas ligand, or a derivative thereof, behind the blood-tissue barrier of the immune privileged site, wherein said Fas ligand fragment, or derivative thereof, has the ability to induce apoptosis in Fas expressing cells.

2. The method according to claim 1, wherein said immune privileged site is selected from the group comprising: the central nervous system (CNS); eye; placenta; testis; and ovaries.

3. The method according to claim 1, wherein said effective amount of the Fas ligand fragment, or derivative thereof, is administered to said animal by a method selected from the group comprising: intrathecal administration; intraventricular administration; and intracistemal administration.

4. The method according to claim 1, wherein said Fas ligand fragment is a recombinant polypeptide.

5. The method according to claim 1, wherein said Fas ligand fragment comprises at least amino acids 103-281 of a human full length Fas ligand.

6. The method according to claim 2, wherein said immune privileged site is the CNS.

7. The method according to claim 6, wherein said inflammation is associated with an autoimmune disorder.

8. The method according to claim 7, wherein said autoimmune disorder is multiple sclerosis.

9. The method according to claim 7, wherein said autoimmune disorder is experimental allergic encephalomyelitis (EAE).

10. The method according to claim 6, wherein said inflammation is associated with a disorder selected from the group comprising: optic neuritis; Devic's disease; encephalitis; myelitis; encephalomyelitis; acute disseminated encephalomyelitis; acute necrotizing hemorrhagic leukoencephalomyelitis; acute transverse myelitis; limbic encephalitis; post-polio syndrome; subacute sclerosing panencephalitis; Guillian-Barre syndrome; acute, subacute, and chronic neuropathy, in which there is radiculitis within the spinal canal; aseptic meningitis; chronic and recurrent meningitis; stroke; CNS trauma; CNS compression; infection; psychiatric diseases; inflammation or rejection after CNS transplantation; neurodenerative diseases; Alzheimer's disease; Parkinson's disease; Huntington's disease; amyotrophic lateral sclerosis; HIV-related encephalopathy; and “stiff-man” syndrome.

11. The method according to claim 2, wherein said immune privileged site is the eye.

12. The method according to claim 11, wherein said inflammation is associated with a disorder selected from the group comprising: uveitis; conjunctivitis; chorioretinitis; uveoretinitis; optic neuritis; intraocular inflammation, such as retinitis and cystoid macular edema; sympathetic ophthalmia; scleritis; retinitis pigmentosa; inflammatory components of degenerative fondus disease; inflammatory components of ocular trauma; ocular inflammation caused by infection; proliferative vitreoretinopathies; acute ischemic optic neuropathy; excessive scarring, for example, following glaucoma filtration operation; and inflammation reaction against ocular implants.

13. The method according to claim 2, wherein said immune privileged site is the testis.

14. The method according to claim 13, wherein said inflammation is associated with a disorder selected from the group comprising: orchitis; epididimo-orchitis; infertility; and orchidal trauma.

15. The method according to claim 1, wherein said animal is a mammal.

16. The method according to claim 15, wherein said mammal is a human.

17. A method of creating an immune privileged site in a tissue of an animal comprising administering an effective amount of Fas ligand fragment comprising the extracellular domain of a full length Fas ligand, or a derivative thereof, behind the blood-tissue barrier of the tissue, wherein said Fas ligand fragment, or derivative thererof, has the ability to induce apoptosis in Fas expressing cells.

18. The method according to claim 17, wherein said animal is a mammal.

19. The method according to claim 18, wherein said mammal is a human.

20. A method of modulating inflammation in an immune privileged site in an animal through the in vivo induction of apoptosis in Fas expressing cells, comprising introducing an effective amount of a Fas ligand fragment comprising the extracellular domain of a full length Fas ligand, or a derivative thereof, behind the blood-tissue barrier of the immune privileged site.

21. The method according to claim 20, wherein said animal is a mammal.

22. The method according to claim 21, wherein said mammal is a human.

23. A method of modulating inflammation within an immune privileged site in an animal by introducing an effective amount of a nucleic acid expressing a Fas ligand fragment comprising the extracellular domain of a full length Fas ligand, behind the blood-tissue barrier of the immune privileged site, wherein said Fas ligand fragment, or derivative thereof, has the ability to induce apoptosis in Fas expressing cells.

24. The method according to claim 23, wherein said immune privileged site is selected from the group comprising: the central nervous system (CNS); eye; placenta; testis; and ovaries.

25. The method according to claim 23, wherein said effective amount of the Fas ligand fragment, or derivative thereof, is administered to said animal by a method selected from the group comprising: intrathecal administration; intraventricular administration; and intracisternal administration.

26. The method according to claim 23, wherein said Fas ligand fragment is a recombinant polypeptide.

27. The method according to claim 23, wherein said Fas ligand fragment comprises at least amino acids 103-281 of a human full length Fas ligand.

28. The method according to claim 24, wherein said immune privileged site is the CNS.

29. The method according to claim 28, wherein said inflammation is associated with an autoimmune disorder.

30. The method according to claim 29, wherein said autoimmune disorder is multiple sclerosis.

31. The method according to claim 29, wherein said autoimmune disorder is experimental allergic encephalomyelitis (EAE).

32. The method according to claim 28, wherein said inflammation is associated with a disorder selected from the group comprising: optic neuritis; Devic's disease; encephalitis; myelitis; encephalomyelitis; acute disseminated encephalomyelitis; acute necrotizing hemorrhagic leukoencephalomyelitis; acute transverse myelitis; limbic encephalitis; post-polio syndrome; subacute sclerosing panencephalitis; Guillian-Barre syndrome; acute, subacute, and chronic neuropathy, in which there is radiculitis within the spinal canal; aseptic meningitis; chronic and recurrent meningitis; stroke; CNS trauma; CNS compression; infection; psychiatric diseases; inflammation or rejection after CNS transplantation; neurodenerative diseases; Alzheimer's disease; Parkinson's disease; Huntington's disease; amyotrophic lateral sclerosis; HIV-related encephalopathy; and “stiff-man” syndrome.

33. The method according to claim 24, wherein said immune privileged site is the eye.

34. The method according to claim 33, wherein said inflammation is associated with a disorder selected from the group comprising: uveitis; conjunctivitis; chorioretinitis; uveoretinitis; optic neuritis; intraocular inflammation, such as retinitis and cystoid macular edema; sympathetic ophthalmia; scleritis; retinitis pigmentosa; inflammatory components of degenerative fondus disease; inflammatory components of ocular trauma; ocular inflammation caused by infection; proliferative vitreoretinopathies; acute ischemic optic neuropathy; excessive scarring, for example, following glaucoma filtration operation; and inflammation reaction against ocular implants.

35. The method according to claim 24, wherein said immune privileged site is the testis.

36. The method according to claim 35, wherein said inflammation is associated with a disorder selected from the group comprising: orchitis; epididimo-orchitis; infertility; and orchidal trauma.

37. The method according to claim 23, wherein said animal is a mammal.

38. The method according to claim 37, wherein said mammal is a human.

39. A method of creating an immune privileged site in a tissue of an animal comprising administering an effective amount of Fas ligand fragment comprising the extracellular domain of a full length Fas ligand, or a derivative thereof, behind the blood-tissue barrier of the tissue, wherein said Fas ligand fragment, or derivative thererof, has the ability to induce apoptosis in Fas expressing cells.

40. The method according to claim 39, wherein said animal is a mammal.

41. The method according to claim 40, wherein said mammal is a human.

42. A method of modulating inflammation in an immune privileged site in an animal through the in vivo induction of apoptosis in Fas expressing cells, comprising introducing an effective amount of a Fas ligand fragment comprising the extracellular domain of a full length Fas ligand, or a derivative thereof, behind the blood-tissue barrier of the immune privileged site.

43. The method according to claim 42, wherein said animal is a mammal.

44. The method according to claim 43, wherein said mammal is a human.

45. A method of modulating inflammation within an immune privileged site in an animal comprising the steps of:
(a) transforming cells in vitro with a nucleic acid encoding a Fas ligand fragment comprising the extracellular domain of a full length Fas ligand;
(b) selecting the cells transformed in step (a) that express the Fas ligand fragment;
(c) introducing the cells selected in step (b) behind the blood-tissue barrier of the immune privileged site,
(d) wherein said Fas ligand fragment, or derivative thereof, has the ability to induce apoptosis in Fas expressing cells.

46. The method according to claim 45, wherein said animal is a mammal.

47. The method according to claim 46, wherein said mammal is a human.
